(19) United States
(12) Patent Application Publication
(10) Pub. No.: US 2007/0066984 A1
(43) Pub. Date: Mar. 22, 2007

(54) Ear cleaning device

(76) Inventor: Ralph Kennedy, Smithtown, NY (US)

Correspondence Address: Mitchell A. Stein, Esq.;STEIN LAW, P.C., Suite 4, 24 Woodbine Avenue, Northport, NY, 11768, US

(21) Appl. No.: 11/231,481

(22) Filed: Sep. 21, 2005

(51) Int. Cl. A61B 17/24

(52) U.S. Cl. 606/161

(57) ABSTRACT

An ear was removal device, having a rod-shaped elongated member, and at least one concave, dome shaped scraping end rigidly affixed to the elongated member of a size sufficient to permit insertion into the ear canal without causing injury to the ear and without pushing a substantial quantity of ear wax further into the ear canal. The ear removal device further has, an option, a cotton swabbing member. The ear removal device can also include two scraping ends each affixed to one end a piece of the rod-shaped elongated member. The member can be comprised of a polymeric substance and/or a cottononous substance. Also shown is a method for ear wax removal, in which the rod-shaped elongated member is inserted into the ear canal, the member has at least one concave, dome shaped scraping end rigidly affixed to the elongated member of a size sufficient to permit insertion into the ear canal without causing injury to the ear and without pushing a substantial quantity of ear wax further into the ear canal, scraping ear wax from the canal using at least one of the novel ends; and removing the device with ear wax thereupon.

### FIELD OF THE INVENTION

The present invention relates to the field of ear cleaning and more particularly to devices inserted into the ear canal for the removal of ear wax through, in this instance, a scraping rather than traditional rubbing devices.

### BACKGROUND OF THE INVENTION

The instant invention relates to ear wax removal. Known in the art are the typical “q-tip” devices which generally all suffer from the same impediments. In particular, these devices generally push the ear wax further into the ear, because the configuration does not provide for simplified removal.

For example, U.S. Pat. No. 5,632,756 shows an ear cleaning device having a bulbous-banded cage assembly which complicates ear removal and in no manner suggests the instant invention. U.S. Pat. No. Des. 358,646 appears to show a brush and point assembly which suggest, quite frankly, more harm than good. U.S. Pat. No. 6,155,987 shows an funnel-shaped speculum for pouring material into the ear canal for removal. In distinction, the subject invention should prevent the need for using any such poured-in solutions for ear wax removal. U.S. Pat. No. 5,176,654 shows a complicated device for inserting fluid into the ear canal again for ear removal. U.S. Pat. No. 4,895,875 describes a peroxide solution, again used for ear wax removal, and generally unnecessary when the subject invention is employed. U.S. Pat. No. 6,417,179 shows another solution.

It is thus an object of the instant invention to provide for a scraping of ear wax from the ear canal rather further insertion of said ear wax, or spreading said ear wax to the canal, or the use of fluids to remove ear wax.

### SUMMARY OF THE INVENTION

The various features of novelty which characterize the invention are pointed out with particularity in the claims annexed to and forming a part of the disclosure. For a better understanding of the invention, its operating advantages, and specific objects attained by its use, reference should be had to the drawings and descriptive matter in which there are illustrated and described preferred embodiments of the invention.

The foregoing objects and other objects of the invention are achieved through an ear removal device, having a rod-shaped elongated member. The rod shaped elongated member is configured so that a user can grab the member for use in scraping ear wax from the ear canal.

Thus, the member has at least one concave, dome shaped scraping end rigidly affixed of a size sufficient to permit insertion into the ear canal without causing injury to the ear and without pushing a substantial quantity of ear wax further into the ear canal. It is critical to the invention that the device have the specific configuration shown in order to prevent pushing ear was further into the ear canal which so typifies existing devices. Thus, the configuration appears dome shaped and is comprised of a compressible material, as defined hereinbelow, in order to prevent pushing ear wax further into the canal, while allowing for expansion once in the canal into the configuration shown in FIG. 1 which provides for scraping and renders the device clearly distinguishable from devices heretofore known.

The ear removal device further has, an option, a cotton swabbing member. In this manner, for those believing in the traditional “q-tip” approach, such persons may have the ability to utilize both the instant device as well as those heretofore known and used by others.

The ear removal device can also include two scraping ends each affixed to one end a piece of the rod-shaped elongated member. In this manner, one device can be used for each ear, thus saving money.

The member can be comprised of a polymeric substance and/or a cottononous substance. Such substances should cause no injury to the ear canal, while allowing for ease of use and expansion once in the canal.

Also shown is a method for ear wax removal, in which the rod-shaped elongated member is inserted into the ear canal, the member has at least one concave, dome shaped scraping end rigidly affixed to the elongated member of a size sufficient to permit insertion into the ear canal without causing injury to the ear and without pushing a substantial quantity of ear wax further into the ear canal, scraping ear wax from the canal using at least one of the novel ends; and removing the device with ear wax thereupon.

Other features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### BRIEF DESCRIPTION OF THE DRAWINGS

In the drawings, wherein similar reference characters denote similar elements through the several views:

FIG. 1 is an overall view of the ear cleaning device in accordance with a preferred embodiment of the subject invention.

### DETAILED DESCRIPTION OF THE PREFERRED EMBODIMENTS

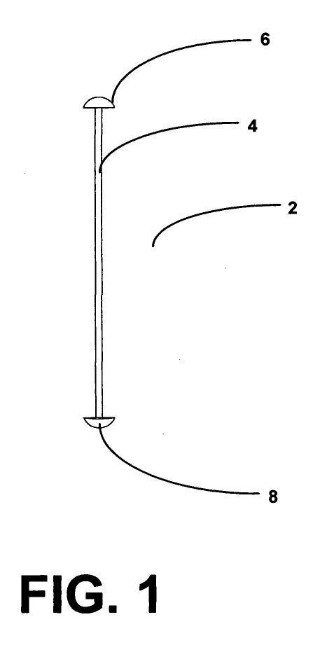

In accordance with the subject invention, FIG. 1 shows an overall view of device 2 of the subject invention having rod 4 with scraping ends 6 and 8 which are physically attached to either end of rod 2. It should be appreciated by one of ordinary skill in the art that the composition of rod 2 may be comprised of plastic, wood, cardboard or any other known material that provides both strength for ear wax scraping, in accordance with the preferred embodiment, as well as flexibility for insertion and extraction into and out of the ear canal.

Ends 6 or 8 are rendered of a flexible, compressible material such that they can be inserted into the ear canal without pushing the ear wax further therein, and without causing injury to the user's ear or canal. Such materials include without limitation, a polymeric or other material that while compressible for insertion rigidifies upon insertion such that scraping, as explained in greater detail hereinbelow, occurs.

It should also be appreciated that while FIG. 1 shows ends 6 and 8, only one need be provided to achieve the fundamental purpose envisioned by the invention. An end other than one having end 6 or 8 can be comprised of any known material including, by way of example, cotton or synthetics utilized by the “q-tip” styled devices known in the prior art.

In accordance with operation, the device 2, shown in FIG. 1 is operated in the following manner. Either end 6 or 8 is inserted by one using rod 4 into the ear canal. The special and unique configuration of ends 6 and 8 provide for easy insertion and compression without pushing the ear wax further into the ear, as is the case in the devices heretofore shown by others. Once inserted, end 6 or 8, having thereby been inserted, extend outwardly into the configuration shown in FIG. 1. As can thereby be observed, these ends 6 and 8 are then configured to scrape the ear wax from the ear canal and remove the same. Accordingly, after insertion and expansion of ends 6 and 8, the user merely twists and pulls, and the ear wax is retrieved, scraped from the canal, and removed as device 2 is extracted from the ear canal.

While there have been shown, described and pointed out fundamental novel features of the invention as applied to preferred embodiments thereof, it will be understood that various omissions and substitutions and changes in the form and details of the device illustrated and in its operation may be made by those skilled in the art without departing from the spirit of the invention. It is the intention, therefore, to be limited only as indicated by the scope of the claims appended hereto.

I claim:

1. An ear was removal device, comprising:
(a) a rod-shaped elongated member; and
(b) at least one concave, dome shaped scraping end rigidly affixed to the elongated member of a size sufficient to permit insertion into the ear canal without causing injury to the ear and without pushing a substantial quantity of ear wax further into the ear canal.

2. The ear removal device of claim 1, further comprising a cotton swabbing member.

3. The ear removal device of claim 1, wherein two scraping ends are included, each affixed to one end a piece of said rod-shaped elongated member.

4. The device of claim 1, wherein said at least one member is comprised of a polymeric substance.

5. The device of claim 1, wherein said at least one member is comprised of a cottononous substance.

6. A method for ear wax removal, comprising the steps of:
(a) inserting a rod-shaped elongated member into the ear canal, wherein said member comprises at least one concave, dome shaped scraping end rigidly affixed to the elongated member of a size sufficient to permit insertion into the ear canal without causing injury to the ear and without pushing a substantial quantity of ear wax further into the ear canal;
(b) scraping ear wax from the canal using said at least one end; and
(c) removing said device with ear wax thereupon.

7. The ear wax removal method of claim 6, further comprising a cotton swabbing member.

8. The ear wax removal method of claim 6, wherein two scraping ends are included, each affixed to one end a piece of said rod-shaped elongated member.

9. The ear wax removal method of claim 6, wherein said at least one member is comprised of a polymeric substance.

10. The device of claim 6, wherein said at least one member is comprised of a cottononous substance.
